(19) United States
(12) Patent Application Publication
(10) Pub. No.: US 2016/0237054 A1
(43) Pub. Date: Aug. 18, 2016

(54) PROCESS FOR THE PURIFICATION OF DAPAGLIFLOZIN

(71) Applicant: SUN PHARMACEUTICAL INDUSTRIES LIMITED, Mumbai, Maharashtra (IN)

(72) Inventors: Suresh Babu JAYACHANDRA, Hyderabad (IN); Devendra Prakash NAGDA, Udaipur (IN); Tarun Kumar SINGH, Lucknow (IN)

(73) Assignee: SUN PHARMACEUTICAL INDUSTRIES LIMITED, Mumbai, Maharashtra (IN)

(21) Appl. No.: 15/025,019

(22) PCT Filed: Sep. 19, 2014
(86) PCT No.: PCT/IB2014/064676

§ 371 Date: Mar. 25, 2016

(30) Foreign Application Priority Data
Sep. 27, 2013 (IN) 2879/DEL/2013

(51) Int. Cl. C07D 309/10

(52) U.S. Cl. CPC C07D 309/10

(57) ABSTRACT

The present invention provides a process for the preparation of (1C)-2,3,4,6-tetra-O-acetyl-1,5-anhydro-1-[4-chloro-3-(4-ethoxybenzyl)phenyl]-D-glucitol of Formula III. The invention also provides a process for the purification of dapagliflozin using (1C)-2,3,4,6-tetra-O-acetyl-1,5-anhydro-1-[4-chloro-3-(4-ethoxybenzyl)phenyl]-D-glucitol of Formula III.

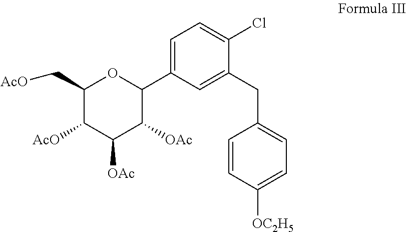 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]

### FIELD OF THE INVENTION

The present invention provides a process for the preparation of (1C)-2,3,4,6-tetra-O-acetyl-1,5-anhydro-1-[4-chloro-3-(4-ethoxybenzyl)phenyl]-D-glucitol of Formula III. The invention also provides a process for the purification of dapagliflozin using (1C)-2,3,4,6-tetra-O-acetyl-1,5-anhydro-1-[4-chloro-3-(4-ethoxybenzyl)phenyl]-D-lucitol of Formula III.

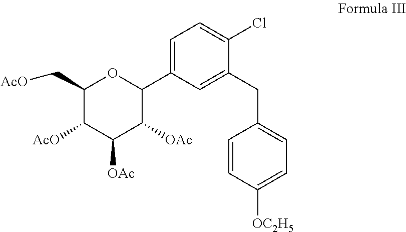 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]

### BACKGROUND OF THE INVENTION

Dapagliflozin propanediol monohydrate is chemically designated as (1S)-1,5-anhydro -1-C-[4-chloro-3-[(4-ethoxyphenyl)methyl]phenyl]-D-glucitol, (S)-propylene glycol, monohydrate and is marketed for the treatment of type 2 Diabetes mellitus. Its chemical structure is represented by Formula I.

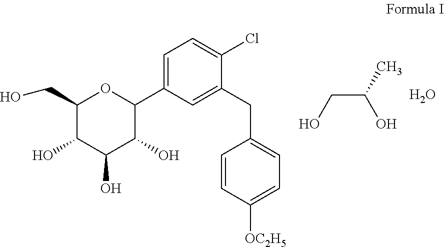

U.S. Pat. Nos. 6,515,117, 7,375,213, 7,932,379, and 7,919,598 disclose processes for the purification of dapagliflozin, comprising the step of acetylating crude dapagliflozin to (1C)-2,3,4,6-tetra-O-acetyl-1,5-anhydro-1-[4-chloro-3-(4-ethoxybenzyl)phenyl]-D-glucitol of Formula III in pyridine. Pyridine is toxic and therefore its use as a solvent should be avoided for industrial production of a pharmaceutical ingredient. Thus, there is a need in the art to develop a process for the preparation of dapagliflozin that avoids the use of pyridine as a solvent.

The present invention provides an improved process for the preparation of the compound of Formula III which circumvents the use of pyridine.

### SUMMARY OF THE INVENTION

A first aspect of the present invention provides a process for the preparation of a compound of Formula III

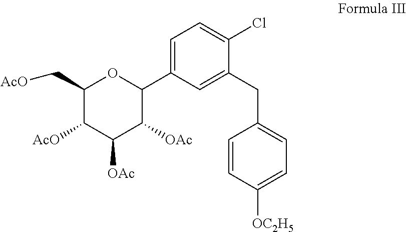 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]

comprising acetylating dapagliflozin of Formula II in a solvent,

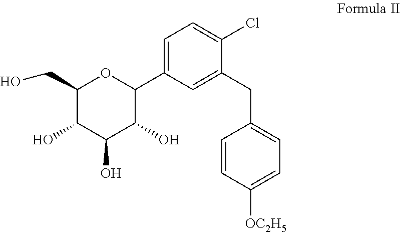 [SMILES: CCOc1ccc(Cc2cc(C3O[C@H](CO)[C@@H](O)[C@H](O)[C@H]3O)ccc2Cl)cc1]

wherein the acetylation is carried out in the absence of pyridine.

A second aspect of the present invention provides a process for the purification of dapagliflozin of Formula II, or solvates thereof, wherein the process comprises the steps of:

a) acetylating dapagliflozin of Formula II in a solvent to obtain a compound of Formula III, wherein the acetylation is carried out in the absence of pyridine; and

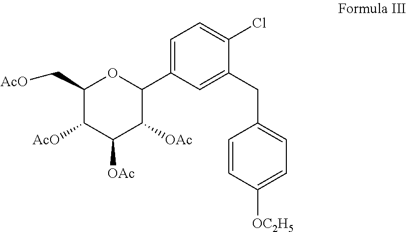 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]

b) deacetylating the compound of Formula III.

### DETAILED DESCRIPTION OF THE INVENTION

The term “about”, as used herein, refers to any value which lies within the range defined by a number up to ±10% of the value.

In the context of the present invention, “solvates” refers to complexes of dapagliflozin with water, methanol, ethanol, n-propanol, propanediol, and butynediol.

The term “acetylation”, as used herein, refers to the addition of acetyl group(s) to a given compound. This can be performed by a reaction of the compound with acetylating agents selected from a group comprising of acetic anhydride, acetyl chloride, and the like.

The term “deacetylation”, as used herein, refers to the removal of acetyl group(s) from a given compound.

The acetylation of dapagliflozin is performed in a solvent selected from a ketone or a chlorinated solvent. Examples of ketone solvents include acetone, methyl ethyl ketone, methyl isobutyl ketone, diisoproyl ketone, methylisopropyl ketone, methylphenyl ketone, and mixtures thereof. Examples of chlorinated solvents include dichloromethane, chloroform, carbon tetrachloride, dichloroethane, and mixtures thereof.

In an embodiment of the present invention, the acetylation of dapagliflozin of Formula II is performed in the presence of a catalyst. Examples of catalysts include dimethylaminopyridine, N-methylpiperazine, copper triflate Cu(OTf)₂, copper(II) tetrafluroborate, phosphomolybdic acid (PMA), and the like

In another embodiment of the present invention, the deacetylation is performed in the presence of a base. Examples of bases include lithium hydroxide, sodium hydroxide, and the like.

In general, acetylation of dapagliflozin is performed using acetic anhydride in the presence of a catalytic amount of dimethylaminopyridine in acetone or dichloromethane to obtain the compound of Formula III, which upon deacetylation with a base gives dapagliflozin.

The preparation of dapagliflozin, which is used as the starting material to prepare the compound of Formula III, is carried out by following the processes described in U.S. Pat. Nos. 6,515,117, 7,375,213, 7,932,379, and 7,919,598, which are incorporated herein by reference.

### Methods

The HPLC purity of dapagliflozin was determined using a Purospher STAR RP-18e (150×4.6 mm), 3 μm column with a flow rate 1.0 mL/minute to 1.5 mL/minute (flow gradient and organic gradient); column oven temperature: 25° C.; sample tray temperature: 25° C.; detector: UV at 225 nm; injection volume: 10 μL; run time: 60 minutes.

The examples below are set forth to aid the understanding of the invention but are not intended to and should not be construed to limit its scope in any way.

### EXAMPLES

### Example 1A

### Preparation of (1C)-2,3,4,6-tetra-O-acetyl-1,5-anhydro-1-[4-chloro-3-(4-ethoxybenzyl)phenyl]-D-glucitol (Formula III)

Acetic anhydride (11.6 mL) was added to dapagliflozin (10 g) in acetone (50 mL) at about 25° C. The reaction mixture was cooled to about 20° C. and dimethylaminopyridine (0.15 g) was added to it at 20° C. to 25° C. The reaction mixture was stirred for about 3 hours at 25° C. to 30° C. After completion of the reaction, the reaction mixture was concentrated under vacuum at 40° C. to 45° C. to obtain a residue. The residue was dissolved in dichloromethane (50 mL) and washed with water (50 mL). The organic layer was separated and concentrated under vacuum to obtain a residue. The residue was dissolved in ethanol (20 mL) and again concentrated at 50° C. to 55° C. to obtain a residue. The residue was dissolved in ethanol (100 mL) and heated to 70° C. to 75° C. to obtain a clear solution. The solution was slowly cooled to about 20° C. and stirred for one hour at 15° C. to 20° C. to obtain a solid. The solid was filtered, washed with ethanol (10 mL), and dried under vacuum at 40° C. to 45° C. for about 16 hours to obtain (1C)-2,3,4,6-tetra-O-acetyl-1,5-anhydro-1-[4-chloro-3-(4-ethoxybenzyl)phenyl]-D-glucitol.

Yield: 6.4 g

Purity: 95.42% (HPLC)

### Example 1B

### Preparation of (1C)-2,3,4,6-tetra-O-acetyl-1,5-anhydro-1-[4-chloro-3-(4-ethoxybenzyl)phenyl]-D-glucitol (Formula III)

Acetic anhydride (7 mL) was added to dapagliflozin (5 g) in acetone (50 mL) at about 25° C. The reaction mixture was cooled to about 20° C. and dimethylaminopyridine (0.15 g) was added to it at 20° C. to 25° C. The reaction mixture was stirred for about 5 hours at 50° C. to 55° C. After completion of the reaction, the reaction mixture was concentrated under vacuum at 40° C. to 45° C. to obtain a residue. The residue was dissolved in ethanol:water (1:1) (50 mL) at 70° C. to 75° C. and gradually cooled to 5° C. to 10° C. and stirred for 1 hour at the same temperature. The solid was filtered, washed with ethanol:water (1:1) (5 mL), and the solid was again dissolved in ethanol (50 mL) at 70° C. to 75° C., gradually cooled to 5° C. to 10° C., and stirred for 1 hour at the same temperature. The solid was filtered, washed with ethanol (5 mL), and dried under vacuum at 40° C. to 45° C. for about 12 hours to obtain (1C)-2,3,4,6-tetra-O-acetyl-1,5-anhydro-1-[4-chloro-3-(4-ethoxybenzyl)phenyl]-D-glucitol.

Yield: 1.5 g

### Example 1C

### Preparation of (1C)-2,3,4,6-tetra-O-acetyl-1,5-anhydro-1-[4-chloro-3-(4-ethoxybenzyl)phenyl]-D-glucitol (Formula III)

Acetic anhydride (11.6 mL) was added to dapagliflozin (10 g) in dichloromethane (100 mL) at about 25° C. The reaction mixture was cooled to about 20° C. and dimethylaminopyridine (0.3 g) was added to it at 20° C. to 30° C. The reaction mixture was stirred for about 3 hours at 40° C. to 50° C. After completion of the reaction, the reaction mixture was cooled to about 30° C. Water (100 mL) was added to the reaction mixture at the same temperature. The layers were separated. The organic layer was concentrated under vacuum at 40° C. to 45° C. to obtain a solid residue. The residue was dissolved in ethanol (20 mL) and the reaction mixture was concentrated under vacuum at 40° C. to 45° C. to obtain a residue. The residue was again dissolved in ethanol (100 mL) at 70° C. to 75° C. The reaction mixture was gradually cooled to about 20° C. and stirred at the same temperature for an hour to obtain a solid. The solid was filtered, washed with ethanol (10 mL), and dried under vacuum at 40° C. to 45° C. for about 12 hours to obtain (1C)-2,3,4,6-tetra-O-acetyl-1,5-anhydro-1-[4-chloro-3-(4-ethoxybenzyl)phenyl]-D-glucitol.

HPLC purity: 98.63%

### Example 2

### Preparation of Dapagliflozin (Formula II)

Lithium hydroxide (0.25 g) was added to a solution of (1C)-2,3,4,6-tetra-O-acetyl-1,5-anhydro-1-[4-chloro-3-(4-ethoxybenzyl)phenyl]-D-glucitol (Formula III; 2.5 g) in methanol (12 mL), tetrahydrofuran (8 mL), and water (4 mL) at 20° C. to 25° C. and stirred for about 4 hours. After completion of the reaction, the reaction mixture was concentrated under vacuum at 40° C. to 45° C. The residue was dissolved in ethyl acetate (25 mL) and washed with an aqueous solution of sodium chloride (10%; 2×25 mL). The layers were separated and the organic layer was concentrated under vacuum at 40° C. to 45° C. to obtain an oily residue. Dichloromethane (10 mL) was added to the oily residue and concentrated under vacuum at 40° C. to 45° C. to obtain dapagliflozin.

We claim:

1. A process for the preparation of a compound of Formula III 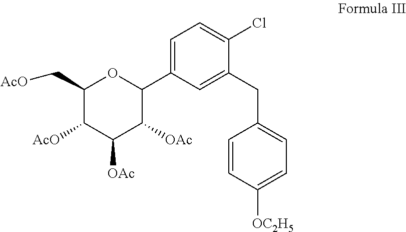 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]
comprising acetylating dapagliflozin of Formula II in a solvent, 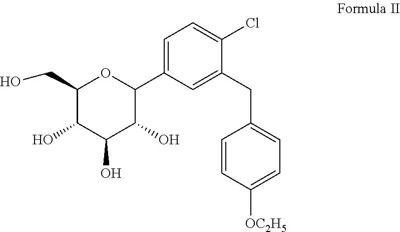 [SMILES: CCOc1ccc(Cc2cc(C3O[C@H](CO)[C@@H](O)[C@H](O)[C@H]3O)ccc2Cl)cc1]
wherein the acetylation is carried out in the absence of pyridine.

2. A process for the purification of dapagliflozin of Formula II, or solvates thereof, wherein the process comprises the steps of:
a) acetylating dapagliflozin of Formula II in a solvent to obtain a compound of Formula III, wherein the acetylation is carried out in the absence of pyridine; and 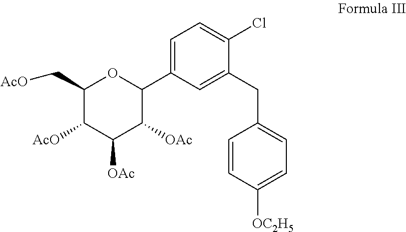 [SMILES: CCOc1ccc(Cc2cc(C3O[C@H](COC(C)=O)[C@@H](OC(C)=O)[C@H](OC(C)=O)[C@H]3OC(C)=O)ccc2Cl)cc1]
b) deacetylating the compound of Formula III.

3. The process according to claim 1 or claim 2, wherein the solvent is selected from a ketone solvent or a chlorinated solvent.

4. The process according to claim 3, wherein the ketone solvent is selected from the group consisting of acetone, methyl ethyl ketone, methyl isobutyl ketone, diisoproyl ketone, methylisopropyl ketone, methylphenyl ketone, and mixtures thereof.

5. The process according to claim 3, wherein the chlorinated solvent is selected from the group consisting of dichloromethane, chloroform, carbon tetrachloride, dichloroethane, and mixtures thereof.

6. The process according to claim 4, wherein the ketone solvent is acetone.

7. The process according to claim 5, wherein the chlorinated solvent is dichloromethane.

8. The process according to claim 2, wherein step b) is carried out in the presence of a base.

9. The process according to claim 8, wherein the base is selected from the group consisting of sodium hydroxide and lithium hydroxide.
